(19) United States
(12) Patent Application Publication
(10) Pub. No.: US 2025/0001085 A1
(43) Pub. Date: Jan. 2, 2025

(54) Medicament delivery device with manual needle insertion

(71) Applicant: SHL Medical AG, Zug (CH)

(72) Inventor: Daniel Carlsson, Nacka Strand (SE)

(21) Appl. No.: 18/693,270

(22) PCT Filed: Sep. 15, 2022
(86) PCT No.: PCT/EP2022/075703

§ 371 Date: Mar. 19, 2024

(30) Foreign Application Priority Data
Oct. 1, 2021 (EP) 21200506.0

(51) Int. Cl. A61M 5/315; A61M 5/32

(52) U.S. Cl. CPC A61M 5/31513; A61M 5/3271

(57) ABSTRACT

The present disclosure provides a medicament delivery device including a release button, a delivery member cover, a rotator, and a housing. The rotator is moveable between a first position wherein the release button is limited by the rotator from being sufficiently pushable to cause a triggering of a delivery of a medicament from the device, a second position in which pushing the release button is possible, and a third position in which the release button is once again limited, and in which a re-transition to e.g. the second position by pushing on the delivery member cover is not possible. A corresponding rotator is also provided.

### CROSS-REFERENCE TO RELATED APPLICATIONS

The present application is a U.S. National Phase Application pursuant to 35 U.S.C. § 371 of International Application No. PCT/EP2022/075703 filed Sep. 15, 2022, which claims priority to European Patent Application No. 21200506.0 filed Oct. 1, 2021. The entire disclosure contents of these applications are herewith incorporated by reference into the present application.

### TECHNICAL FIELD

The present disclosure relates to the field of medicament delivery devices. In particular, the present disclosure relates to such a device configured for manual insertion of the needle.

### BACKGROUND

Autoinjectors and other types of medicament delivery devices are designed to allow for a user (e.g. a patient) to perform self-administration of a dose of a particular medicament in a controlled way. After positioning the device at the site of the body wherein the injection is desired, the user activates the device and triggers a delivery of the medicament by pushing a release button. However, in order to reduce the risk of accidental activation, some devices include one or more safety mechanisms that the user must disable before pushing of the release button is possible to trigger the delivery. One such safety mechanism includes providing a delivery member cover mechanically interacting with a so called “rotator”, wherein pushing of the release button to activate the device is not possible unless the rotator is first rotated by pressing the delivery member cover against the injection site. In some devices, the needle is caused to penetrate the injection once the device is activated, while in some other devices the needle is manually inserted by the user during the pushing of the needle cover against the injection site.

In devices relying on such a manual insertion of the needle, situations may arise where the user for example fails to fully insert the needle before attempting to push the release button, or where the user for example does fully insert the needle but then forgets to also press the release button before pulling the needle out of the injection site. In both such situations, problems can arise as the needle would e.g. no longer be sterile even though no delivery of the medicament has taken place, or as a triggering of the device after the needle has been pulled out can be less desirable.

### SUMMARY

To at least partially solve the above identified problem with already available such medicament delivery devices, the present disclosure provides an improved medicament delivery device (i.e. “a device”), and also an improved rotator for such a device as defined in the accompanying independent claims. Various alternative embodiments of the improved device and rotator are defined in the dependent claims.

According to a first aspect of the present disclosure, a medicament delivery device is provided. The device includes a release button, a delivery member cover, a rotator, and a housing. The delivery member cover includes a track and the rotator includes a track follower, or vice versa. The track follower protrudes into the track, and the track includes at least a first track edge and a second track edge tilted with a first angle and a second angle, respectively, with respect to a longitudinal axis of the device. The rotator includes a first blocking member and the release button includes a second blocking member. The delivery member cover is longitudinally moveable and rotationally fixed relative to the housing. The rotator is free to rotate relative to the housing. The release button is longitudinally moveable and rotationally fixed relative to the housing. The rotator is configured to move from a first position to a second position to a third position. The first position, the second position, and the third position, are all rotationally different from one another. In the first position of the rotator, the first track edge is aligned in a longitudinal direction with the track follower. In the second position of the rotator, the second track edge is aligned in the longitudinal direction with the track follower. When the delivery member cover is moved in a distal direction relative to the rotator, the track follower engages the first track edge, thereby rotating the rotator from the first position to the second position in a first rotational direction. When the delivery member cover is subsequently moved back in a proximal direction relative to the rotator, the track follower engages the second track edge, thereby rotating the rotator from the second position to the third position in a second rotational direction. The first and second angles are such that the first and second rotational directions are the same. The first blocking member and the second blocking member are aligned in the first position of the rotator, thereby limiting movement of the release button in the proximal direction relative to the rotator. The first blocking member and the second blocking member are not aligned in the second position of the rotator, thereby allowing movement of the release button in the proximal direction relative to the rotator. The first blocking member and the second blocking member are aligned in the third position of the rotator, thereby limiting movement of the release button in the proximal direction relative to the rotator.

Here, in the first and third positions of the rotator, “limiting movement of the release button” should be understood as that there may be some allowed travel of the release button in the proximal direction, but not enough to cause a triggering of a delivery of a medicament from the device. Likewise, in the second position of the rotator, “allowing movement of the release button” should be understood as that there may still be a limiting of the movement of the release button, but not limiting enough to block the release button from being pushed sufficiently far in the proximal direction to trigger a delivery of a medicament from the device.

In the above, it is envisaged that there may be a single housing provided in which all of the release button, the delivery member cover and the rotator are arranged. It is also envisaged that the housing may be divided into several parts, i.e. sub-housings, and that some of the parts may be arranged in one sub-housing, other parts in a second sub-housing, etc. To allow a part to be moveable in a longitudinal direction, but not rotatable around the longitudinal direction, may for example be achieved by the housing having e.g. a longitudinal groove/track/slot and the part having e.g. a protrusion which engages in the longitudinal groove/track/slot, or vice versa. To allow a part to be rotatable around the longitudinal direction, but not moveable in the longitudinal direction, may for example be achieved by the housing instead having a groove/track/slot which extends circumferentially and the part having e.g. a protrusion which engages in the circumferential groove/track/slot, or vice versa. In particular, any configuration of the release button, the delivery member cover, the rotator and the housing is envisaged which allows for both of the release button and the delivery member cover to move longitudinally with respect to the rotator, and which allows the rotator to be rotated around the longitudinal direction with respect to the release button and the delivery member cover. In some situations, a longitudinal movement or a rotation may however be blocked or limited by other parts of the device, and/or by the relative current alignment of the various parts. However, as long as the device can be operated in any state wherein the parts can move/rotate relative each other, as described above, it is considered that the various parts are still to be considered as being moveable and/or rotatable relative to each other.

As generally envisaged herein, a longitudinal movement of a part may be caused directly by e.g. a user pushing the part in question (by applying a linear force thereon), or be caused indirectly if the linear force is instead applied by another part of the device. Likewise, a rotation of a part relative to another can be caused either by a user directly rotating the part (by applying a torque thereon), or be caused indirectly if the torque is instead applied by another part of the device. In general, that some part is “pushed”/“moved” or “rotated” is to be understood as if the part in question is “pushed”/“moved” or “rotated” with respect to some other part of the device. In what follows, “applying a force on” and “pressing” a part will be used interchangeably. It should be noted that pressing a part may not necessarily cause the part to move or be pushed, as the part may currently be blocked by e.g. some other part of the device.

Herein, when it is referred to that the rotator is in a specific position (such as the first position, the second position and/or the third position), it is understood that rotator then has a certain rotational relative alignment with respect to some other part. The first, second and third positions of the rotator may for example imply that the rotator has a first, second and third rotational relative alignment with respect to the delivery member cover, and/or with respect to the release button.

A change in the position of the rotator is to be understood as including a rotation of the rotator vis-à-vis e.g. the release button and/or the delivery member cover. Consequently, it is such rotations which cause the rotator to move from one position to another, as long as the rotation is large enough.

The present disclosure according to the first aspect can improve upon existing technology in that the device as disclosed herein thus both prevents an unwanted triggering of the device by sufficiently blocking/limiting the release button (from causing a triggering of a delivery of a medicament from the device) until the delivery member cover is first sufficiently pushed (against the site of injection), but also in that after the delivery member cover is released (independent of whether the release button was actually sufficiently pushed or not), the release button is once again blocked/limited from triggering the device. Consequently, once the delivery member cover has been pushed and released, the user has no additional chance of triggering the device (provided that the push/movement of the delivery member cover was greater than e.g. a predefined threshold, which may or may not be finite). As will be described later herein, the envisaged device can be of a manual type wherein the user inserts the needle into an injection site by pushing the delivery member cover against the injection site, and only thereafter triggers the delivery of the medicament through the needle by pushing the release button. This may prevent a situation as described earlier herein, wherein triggering of the device is possible even if the needle has already been withdrawn from the injection site.

In one or more embodiments of the device, in the first position of the rotator, the track follower may be closer to a proximal end of the device than the first track edge. In the second position of the rotator, the track follower may be closer to a distal end of the device than the second track edge. This may be particularly so if the track follower forms part of the delivery member cover, while the track forms part of the rotator.

In one or more embodiments of the device, in the third position of the rotator, the delivery member cover may be blocked from being moved in the distal direction relative to the rotator. In addition to preventing the user from triggering the device by once again attempting to push on the delivery member cover, blocking (i.e. locking) the delivery member cover in this state (for example, as fully or almost fully released) may also have the advantage that the delivery member cover reduces the risk of the user coming into contact with the delivery member (e.g. the needle), either directly or after attempting to once again push on the delivery member cover. In particular, this can be important as the needle may have entered the injection site and is no longer sterile.

In one or more embodiments of the device, the delivery member cover may include at least one locking arm. The at least one locking arm may be configured to, in the third position of the device, interact with a part of the device (such as a locking ledge or similar) other than the delivery member cover to block the delivery member cover from being (re-) pushed. This may provide one way of obtaining the effect described earlier herein, wherein the delivery member cover is blocked after it is first pushed and then released. The locking arm may for example interact with a body/cover/housing of the device. The locking arm may for example include a hull or similar, which hooks into a corresponding recess or ledge of (the body of) the device, such that the delivery member cover may extend e.g. fully away from the rotator but not move back in the opposite direction.

In one or more embodiments of the device, in the third position of the rotator, neither the first track edge nor the second track edge may be aligned in the longitudinal direction with the track follower. This may further help to prevent that an attempt to once again push on the delivery member cover causes the rotator to rotate such that it no longer limits the release button from being sufficiently pushable to trigger the device.

In one or more embodiments of the device, the delivery member cover may be spring loaded such that when the delivery member is moved in the distal direction relative to the rotator, potential spring energy is built up, and such that when the delivery member cover is subsequently moved back in the proximal direction relative to the rotator, this is at least partly driven by a release of the (built-up) potential spring energy. Using a spring-loaded arrangement can for example provide a suitable force which must be overcome in order to push the delivery member cover, and also help to release the delivery member cover after being pushed without solely having to rely on e.g. gravity. The exact dimensions of the spring(s) can be tailored according to particular needs and desires, and for example be made different for devices targeting different users if wanted.

In one or more embodiments of the device, the first blocking member may include a protrusion and the second blocking member include at least one edge including a recess, or vice versa. In the first and third positions of the rotator, the protrusion may rotationally align with the at least one edge but not with the recess. In the second position of the rotator, the protrusion may rotationally align with the recess. As will be explained herein, aligning the recess with the protrusion may offer a longer possible travel distance when pushing the release button in the proximal direction, such that the release button may trigger the device. Not aligning the protrusion with the recess, such that the protrusion instead abuts the at least one edge if the release button is pushed, may thus reduce the possible travel distance when pushing the release button in the proximal direction, such that the release button may not trigger the device.

In one or more embodiments of the device, the at least one edge may be at a same position relative to the longitudinal axis on both sides of the recess.

In one or more embodiments of the device, the device may further include means for either directly or indirectly holding a delivery member (in form of e.g. a needle) for the delivery of the medicament, and configured such that the delivery member can be manually inserted into a body by a user of the device moving the delivery member cover in the distal direction relative to the rotator by pressing the delivery member cover against an injection site of/on the body. The device may thus be a so-called manual injection device, wherein the needle is not forced into the body by e.g. a spring, when triggering the device, but where the user can insert the needle at an own pace and under an own control, with the configuration of the device reducing or avoiding the negative possible outcomes of such a manual injection device (as described herein).

According to a second aspect of the present disclosure, a rotator for a medicament delivery device is provided. It is envisaged that the delivery device includes a release button, a delivery member cover, and a housing (e.g. as described above and later herein). The rotator has a longitudinal axis and includes a track configured to receive a track follower of the delivery member cover, and at least a first track edge and a second track edge tilted with a first angle and a second angle, respectively, with respect to the longitudinal axis. When the rotator is arranged in the device to freely rotate relative to the housing and with the track follower received in the track, the rotator is configured to move from a first position to a second position to a third position. The first position, the second position, and the third position, are all rotationally different from each other. In the first position of the rotator, the first track edge is aligned in the longitudinal direction with the track follower. In the second position of the rotator, the second track edge is aligned in the longitudinal direction with the track follower. When the delivery member cover is moved in a distal direction relative to the rotator, the track follower engages the first track edge, thereby rotating the rotator from the first position to the second position in a first rotational direction. When the delivery member cover is subsequently moved back in a proximal direction relative to the rotator, the track follower engages the second track edge, thereby rotating the rotator from the second position to the third position in a second rotational direction. The first and second angles are such that the first and second rotational directions are the same.

In one or more embodiments of the rotator, the rotator may include a first blocking member configured to interact with a second blocking member of the release button, to either limit or not limit the movement of the release button in the proximal direction relative to the rotator depending on the position of the rotator. For example, as described above with reference to the device of the first aspect, the rotator may be configured such that the blocking members interact to provide the limiting in the first and third positions of the rotator, but not in the second position of the rotator.

In one or more embodiments of the rotator, the first blocking member may include a protrusion configured to interact with a corresponding at least one edge including a recess of the second blocking member of the release button.

In one or more embodiments of the rotator, the first blocking member may include at least one edge including a recess configured to interact with a corresponding protrusion of the second blocking member of the release button. In this and the previous embodiment described above, such a configuration of protrusions and edges/recesses may offer a convenient way of blocking the release button in some positions of the rotator but not in others.

In one or more embodiments of the rotator, the at least one edge may be at a same position relative to the longitudinal axis of the rotator on both sides of the recess.

Herein, it is envisaged that the various “first”, “second” and “third” “positions of the rotator” of the second aspect correspond to the various “first”, “second” and “third” “states” of the rotator described with reference to the device of the first aspect, and vice versa.

Other objects and advantages of the present disclosure will be apparent from the following detailed description, the drawings and the claims. Within the scope of the present disclosure, it is envisaged that all features and advantages described with reference to e.g. medicament delivery device of the first aspect are relevant for, apply to, and may be used in combination with also the those described with reference to the rotator of the second aspect, and vice versa.

### BRIEF DESCRIPTION OF THE DRAWINGS

Exemplifying embodiments will now be described below with reference to the accompanying drawings, in which:

FIGS. 1A-1C schematically illustrate various states and relative alignments of components in an embodiment of a medicament delivery device according to the present disclosure;

FIGS. 1D and 1E schematically illustrate various other embodiments of medicament delivery devices according to the present disclosure, and

FIGS. 1F and 1G schematically illustrate perspective views of various embodiments of rotators according to the present disclosure.

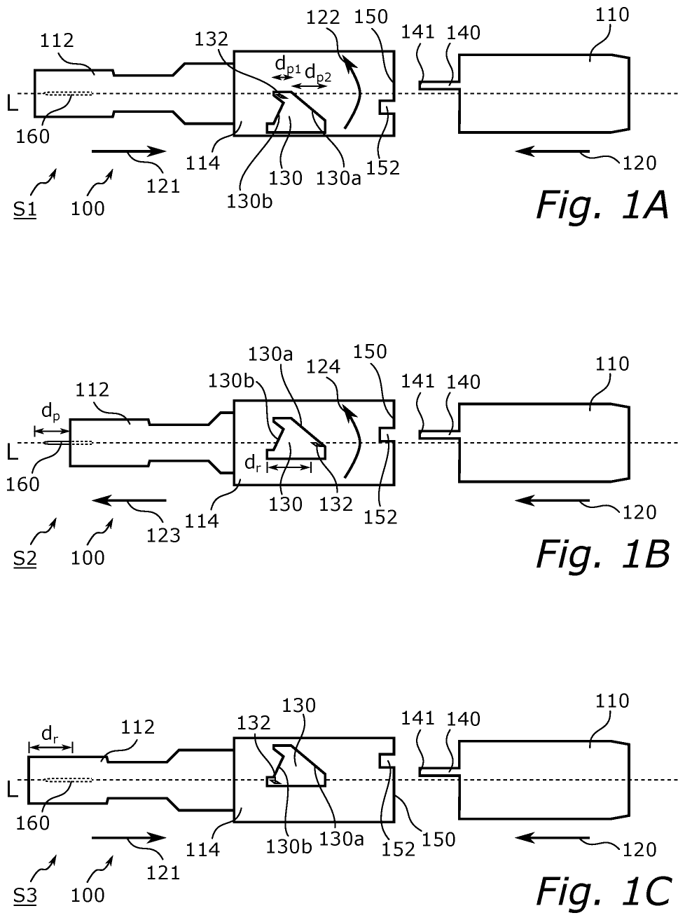

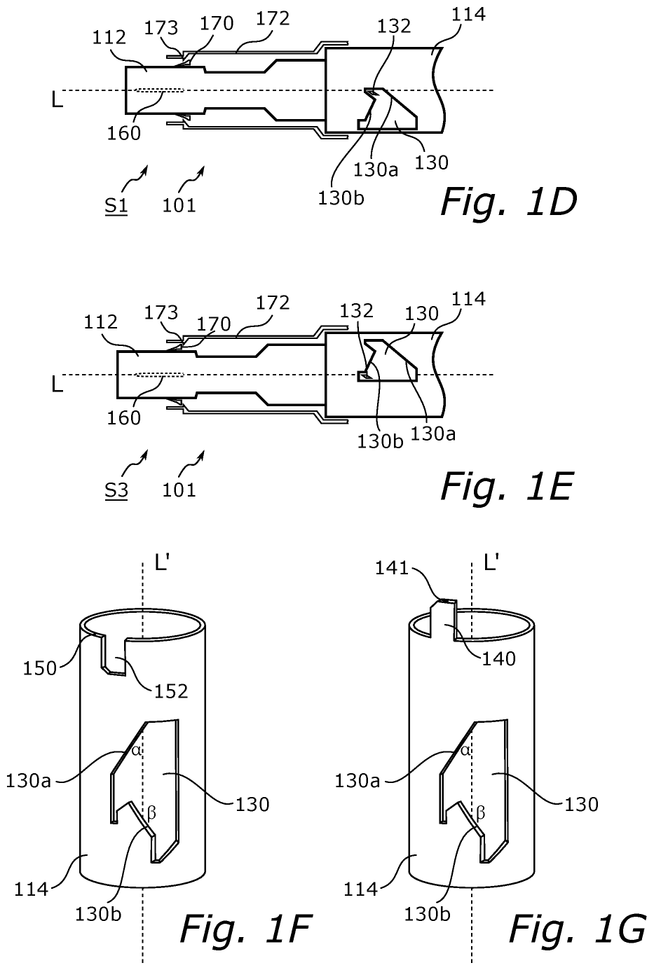

Although described as illustrating a device, FIGS. 1A-1E also serves to illustrate various embodiments of a rotator according to the present disclosure, and its interaction with the device as a whole. Likewise, although described as illustrating a rotator, FIGS. 1F-1G also serves to illustrate various embodiments of a device according to the present disclosure, in which such rotators is envisaged as being usable.

In the drawings, like reference numerals will be used for like elements unless stated otherwise. Unless explicitly stated to the contrary, the drawings show only such elements that are necessary to illustrate the example embodiments, while other elements, in the interest of clarity, may be omitted or merely suggested. As illustrated in the Figures, the (absolute or relative) sizes of elements and regions may be exaggerated or understated vis-à-vis their true values for illustrative purposes and, thus, are provided to illustrate the general structures of the embodiments.

### DETAILED DESCRIPTION

Exemplifying embodiments of a medicament delivery device (and a rotator for such a device) according to the present disclosure will now be described more fully hereinafter with reference to the accompanying drawings. The drawings show currently preferred embodiments, but the invention may, however, be embodied in many different forms and should not be construed as limited to the embodiments set forth herein; rather, these embodiments are provided for thoroughness and completeness, and fully convey the scope of the present disclosure to the skilled person.

The various components of a device (including a rotator) as envisaged herein, as well as the various states in which the device (and rotator) can be operated, will now be described with reference to FIGS. 1A-1C. These Figures illustrate a particular embodiment of a device as envisaged herein.

FIG. 1A schematically illustrates a device 100 which is currently in a first state S1. Generally in what follows, such a “state” corresponds to a particular “position of the rotator” as referred to in the claims. In FIG. 1A, the state S1 corresponds to a rotator 114 of the device 100 being in its first position.

The device 100 further includes a release button 110 which is arranged such that it may be longitudinally moveable but rotationally fixed relative to a housing (not shown) of the device 100. Phrased differently, the release button 110 may be pressed/displaced linearly in a direction 120 along a longitudinal axis L of the device 100. The direction 120 is a proximal direction of the device. The release button 110 may for example be spring loaded by using a spring (not shown) which provides, when the release button 110 is pressed in the direction 120, a counteracting force in a direction opposite to the direction 120. The spring constant of the spring may be selected low enough such that e.g. a user's thumb pressing on the release button 110 is strong enough to overcome the counteracting force provided by such a spring, but high enough such that e.g. shaking of the device 100 does not cause any accidental pushing of the release button 110. To make the release button 110 longitudinally moveable but rotationally fixed relative to the housing, it is envisaged that the housing may for example include a groove (or track, or slot, etc.) which extends longitudinally along the axis L. The release button 110 may have a corresponding protrusion that engages in the groove of the housing, and the groove and protrusion may interact such that movement of the release button 110 is only possible in the longitudinal direction (as the groove would block the protrusion and thus the rotator from moving circumferentially). It is also envisaged that the groove may form part of the release button 110, and that the protrusion which engages the groove is instead a part of the housing.

The device 100 further includes a delivery member cover 112. The delivery member cover 112 may be hollow and shaped as for example a sleeve/cylinder, and functions to cover a delivery member 160 (such as e.g. a needle) from being accessible by a user in the state S1 of the device 100. The delivery member 160 may in turn be connected to e.g. a syringe/ampulla (not shown) located in the device, such that medicament may be injected through the delivery member 160.

The delivery member cover 112 is, just like the release button 110, also arranged in the device 100 such that it is longitudinally moveable but rotationally fixed relative to the housing of the device. Phrased differently, the delivery member cover 112 may be pressed and thereby displaced linearly along the longitudinal axis L, along a direction 121. The direction 121 is a distal direction of the device. The delivery member cover 112 may also be spring loaded by one or more springs (also not shown), which may provide, when the delivery member cover is pressed in the direction 121, a counteracting force in a direction opposite to the direction 121. The spring constant for this spring may be selected low enough such that e.g. a hand of the user is strong enough to overcome the counteracting force when pressing the delivery member cover and the device against e.g. the user's skin, but high enough such that the delivery member cover is not accidentally pressed by e.g. shaking the device 100 or letting the device 100 just rest against the user's skin. Just as for the release button 110, the restriction of rotational movement of the delivery member cover 112 relative to the housing may be achieved by a combination of a groove (or track, or slot, etc.) and protrusion.

The device 100 further includes the rotator 114. The rotator 114 is arranged within the device 100 such that it is free to rotate relative to the housing, i.e. such that it may be rotated around the longitudinal axis L, and thereby rotated also relative to the release button 110 and the delivery member cover 112. The rotator 114 may e.g. be hollow and shaped for example like a sleeve/cylinder. The rotational movement of the rotator 114 relative to the housing may for example be achieved by a combination of a circumferentially extending groove (or track, or slot, etc.) on either one of the housing and the rotator 114, and a corresponding protrusion engaging such a groove and located on the other one of the housing and the rotator 114. This may also prevent the rotator 114 from moving in the longitudinal direction relative to the housing (and also relative the release button 110 and the delivery member cover 112).

The rotator 114 has a track 130 including a first track edge 130a and a second track edge 130b. The delivery member cover 112 has a corresponding track follower 132, which protrudes into the track 130. The first track edge 130a and the second track edge 130b are tilted with a first angle and a second angle, respectively, with respect to the longitudinal axis L of the device. As can be seen from FIG. 1A, in the first state S1 (and the first position of the rotator 114), the first track edge 130a is aligned with the track follower 132. The first track edge 130a is thus arranged such that in the state S1, the track follower 132 of the delivery member cover 112 will eventually engage with and follow the first track edge 130a if the delivery member cover 112 is pushed/moved towards the rotator in the direction 121. This arrangement of the first track edge 130a and the track follower 132 allows a linear displacement of the delivery member cover 112 in the direction 121 to cause a torque 122 being applied to/on the rotator 114. If the rotator 114 is not locked by other means, the applied torque 122 causes a rotation of the rotator 114 around the longitudinal axis L of the device. Phrased differently, the arrangement of the first track edge 130a of the rotator 114 and the track follower 132 of the delivery member cover 112 allows for a linear displacement (i.e. a pushing) of the delivery member cover 112 to be converted into a rotational displacement (i.e. a rotation) of the rotator 114 (relative to e.g. the release button 110). The rotational displacement will cause the rotator 114 to move from the first position into a second position (as will be described below with reference to FIG. 1B).

Herein, that a component is “displaced linearly” is to be understood as said component being displaced linearly relative to one or more other components of the device 100, along the longitudinal axis L, and does not mean e.g. any linear displacement of e.g. the device 100 as a whole. Pushing of the release button 110 may for example mean that the release button 110 moves towards the rotator 114 (and also towards the delivery member cover 112) along the longitudinal axis L. Pushing of the delivery member cover 112 may for example mean that the delivery member cover 112 moves towards the rotator 114 (and also towards the release button 110) along the longitudinal axis L.

The release button 110 is further configured to, if pressed (i.e. linearly displaced) sufficiently in the direction 120, trigger a medicament delivery mechanism (not shown) within the device 100. When triggered, the medicament delivery mechanism can cause an amount of medicament stored within the device (in for example an ampulla/syringe, also not shown) to be ejected through the delivery member 160 (e.g. through a needle). However, in the state S1 of the device (i.e. in the first position of the rotator 114) as illustrated in FIG. 1A, the release button 110 is prevented from being pressed sufficiently to cause such triggering (i.e. the rotator 114 blocks the release button 110 from being sufficiently pushable to trigger the delivery). This is obtained as follows:
I) The release button 110 further includes a second blocking member in form of a protrusion 140 which extends out towards the rotator 114 from a side/surface of the release button 110 arranged towards the rotator 114. The rotator has a corresponding first blocking member in form of at least one edge 150 facing towards the protrusion 140 of the release button 110. The at least one edge 150 includes a recess 152 therein. In the particular embodiment of the device 100 shown in FIGS. 1A-1C, the edge 150 is such that it extends on both sides of the recess 152, and also such that a position of the edge 150 on the longitudinal axis L is the same on both sides of the recess 152.
II) In the state S1 of the device 100, a rotational relative alignment of the release button 110 and the rotator 114 is such that the protrusion 140 of the release button 110 aligns with the edge 150 (but does not align with the recess 152) of the rotator 114. Consequently, the release button 110 may be pressed in the direction 120, but only until an edge 141 of the protrusion 140 abuts/presses against the at least one edge 150 of the rotator 114. The relative possible displacement of the release button 110 so achieved is thus limited and not sufficient to cause the triggering of the medicament delivery mechanism.

However, once the delivery member cover 112 is displaced enough (e.g. a first finite push distance dₚ₁) in the direction 121 for the track follower 132 to engage with and follow the first track edge 130a, the torque 122 thus generated and applied on the rotator 114 can start to cause a rotation of the rotator 114, thereby moving the rotator 114 from its first position into the second position (corresponding to a state S2 of the device which will be described with reference to FIG. 1B). In the particular configuration illustrated in FIGS. 1A-1C, transitioning into the second state S2 by moving the rotator 114 to its second position requires that the delivery member cover 112 is pushed greater than a finite push distance dₚ. The distance dₚ can be tailored by changing e.g. one or both of the arrangement/configurations of the track follower 132 and the arrangement/configuration of the first track edge 130a. The distance dₚ may for example be the sum of the first finite push distance dₚ₁ required before the track follower 132 reaches the first track edge 130a, and a second finite push distance dₚ₂ required for the interaction between the track follower 132 and the first track edge 130a to cause enough rotation of the rotator 114 for the device 100 to enter the second state S2.

The delivery member 160 can be arranged such that it does not move with the delivery member cover 112, but instead stays fixed with respect to e.g. the rotator 114 or e.g. a body (not shown) of the device 100. Thus, during the transitioning to the second state S2 (i.e. when the delivery member cover 112 is pushed), the delivery member 160 is exposed to the user and inserted into the injection site against which the delivery member cover 112 is pressed, as further illustrated in FIG. 1B. This allows a user of the device 100 to manually insert the delivery member 160 by pushing on the delivery member cover 112. Fixing of the delivery member 160 can for example be achieved by blocking its movement in a distal direction by using the rotator 114, and by blocking its movement in a proximal direction by using e.g. a housing/cover (not shown) of the device 100. Other possible solutions are also envisaged.

FIG. 1B schematically illustrates the same device 100 but in the second state S2. In the second state S2, the rotational relative alignment of the release button 110 and the rotator 114 has changed (by the application of the first torque 122) such that the rotator 114 is now in its second position, and such that the recess 152 of the rotator 114 now aligns with the protrusion 140 of the release button 110. Further pushing the release button 110 to eventually or directly cause the triggering of the medicament delivery mechanism is therefore possible, as the allowed travel distance for the release button 110 is now greater than in the state S1. As the delivery member 160 is exposed (and inserted into the injection site), triggering of the device 100 can cause the medicament to be delivered through the delivery member 160 and into the injection site against which the delivery member cover 112 is pushed.

In the state S2 and the second position of the rotator 114, subsequently moving the delivery member cover 112 back in the (proximal) direction 123, by e.g. releasing the delivery member cover 112 (that is, to stop pushing the delivery member cover 112 and instead allowing e.g. gravity force, a spring force, or any other suitable force, to be exerted on the delivery member cover 112 in a direction 123 opposite to the direction 121) will eventually force the track follower 132 to instead engage with and follow the second track edge 130b of the rotator 114. This because in the second position of the rotator 114, the second track edge 130b is aligned in the longitudinal direction with the track follower 132. When the track follower 132 engages with the second track edge 130b, the arrangement of the track follower 132 and the direction of the slope/tilt/angle of the second track edge 130b is such that another torque 124 will be generated and applied on the rotator 114, thus transitioning the device 100 into a third state S3 that will be described with reference to FIG. 1C. In this third state S3, the rotator 114 is in its third position.

In the particular example of a device shown in FIGS. 1A-1C, both the first torque 122 and the second torque 124 causes rotations of the rotator 114 in a same direction. This is achieved, as will be explained later with reference to e.g. FIGS. 1F-1G, due to the angles of the respective first and second track edges 130a and 130b. As can be seen from FIGS. 1A-1C, the tilting angles of the first and second track edges 130a and 130b are such that sign of the first angle (measured from the longitudinal axis) is opposite to the sign of the second angle (measured in the same way). Thus, the track follower 132 pushing on the first track edge 130a in the distal direction 121 will cause a rotation of the rotator 114 in a same direction as when the track follower 132 pushes on the second track edge 130b in the proximal direction 123.

To transition the device 100 into the third state S3, the delivery member cover 112 must in the particular embodiment shown in FIGS. 1A-1C be released greater than a finite release distance dr. The exact size of this distance dr depends on how far the rotator 114 has been rotated, the exact configuration/arrangement of the various track follower 132 and first track edge 130a and second track edge 130b, etc. In the specific embodiment illustrated in FIGS. 1A-1C, the distance dr is greater than the distance dₚ, such that in the third state S3, the delivery member cover 112 is released further than its position in the first state S1. In one or more other embodiments, it is envisaged that the release distance of the delivery member cover 112 in the third state S3 can be the same as, or even smaller than, the release distance of the delivery member cover 112 in the first state S1.

It should be noted that the transitioning of the device 100 into the third state S3 (that is, the moving of the rotator 114 from its second to its third position) can happen even if the delivery member cover 112 is not pushed fully, i.e. even if the delivery member 160 is only partly inserted into the injection site against which the delivery member cover is pushed. This is because of the presence, configuration and arrangement of the second track edge 130b relative the track follower 132 in the second position of the rotator 114.

FIG. 1C schematically illustrates the device 100 in the third state S3. The applied second torque 124 has caused the rotational relative alignment of the release button 110 and the rotator 114 to change such that the rotator 114 is now in its third position, and such that the recess 152 of the rotator 114 once again does not align with the protrusion 140 of the release button 110.

Instead, the edge 150 once again aligns with the protrusion 140, and in the third position and the third state S3, the release button 110 is therefore again blocked/limited by the rotator 114 from being sufficiently pushable to trigger the delivery of the medicament (due to the interaction between the first and second blocking members). Also, in the third state S3 and the third position of the rotator 114, the release of the delivery member cover 112 is such that the delivery member cover 112 once again covers the delivery member 160 and prevents the delivery member 160 from being exposed and possible stinging the user.

It should be noted that in the third state S3 and the third position of the rotator 114, pushing once again on the delivery member cover 112 in the direction 121 could possibly cause a linear movement of the track follower 132, but that there is no track edge 130a and 130b with which the track follower 132 aligns, such that such linear movement could be converted into a torque applied on the rotator 114. Consequently, in the state S3 with the rotator 114 in its third position, a further change of the rotational relative alignment of the release button and the rotator, such that the recess 152 realigns with the protrusion 140 such that the release button 110 is once again sufficiently pushable (i.e. not limited) to cause the triggering, is not possible by pressing the delivery member cover 112 in the direction 121.

An optional configuration of a device 101 will now be described in more detail with reference to FIGS. 1D and 1E.

FIGS. 1D and 1E schematically illustrates parts of another embodiment of a device 101 that is identical to the device 100 described with reference to FIGS. 1A-1C, except that the delivery member cover 112 further includes locking arms 170 which are arranged to interact with another part of the device 101, such as the housing/cover 172. With the device 101 in its first state S1 with the rotator 114 in its first position (as shown in FIG. 1D), the delivery member cover 112 can be pushed towards the rotator 114 as described earlier herein. In the third state S3 (as shown in FIG. 1E), the release of the delivery member cover 112 is greater than in the first state S1, and the locking arms 170 are therefore pushed between locking ledges 173 of the housing/cover 172 and snapped such that the delivery member cover 112 is now blocked from being pushed again towards the rotator 114. This has the benefit that if for example the delivery member 160 is no longer sterile (due to e.g. aborting the pushing of the delivery member cover 112 before the delivery member 160 is fully inserted, or similar), the delivery member cover 112 shields the delivery member 160 such that the risk of the user e.g. accidentally stinging on the delivery member 160 is reduced. It is envisaged that one or both of the locking arms 170 and the locking ledges 173 may be flexible, and made of for example plastic or metal having suitable properties therefor. That the release of the delivery member cover 112 is greater in the third state S3 than in the first state S1 can be achieved e.g. by the arrangement and configuration of the track follower 132 and the various track edges, as described earlier herein with reference e.g. to FIGS. 1A-1C. In other embodiments, there may be either a single locking arm 170 and locking ledge 173, or for example more than two locking arms 170 and locking ledges 173.

The devices described herein so far with reference to FIGS. 1A-1E have the track 130 and various track edges 130a and 130b as part of the rotator 114, and the track follower 132 as part of the delivery member cover 112. In such configurations, in the first position of the rotator 114, the track follower 132 is closer to a proximal end of the device than the first track edge 130a, and in the second position of the rotator 114, the track follower 132 is closer to a distal end of the device than the second track edge 130b. It is, however, envisaged also that the opposite can be the case. Phrased differently, a same or similar advantage as described herein can be achieved by arranging the delivery member cover as a sleeve surrounding the rotator, and by having the track and various track edges as part of the delivery member cover and the track follower (or multiple track followers) as part of the rotator instead.

A rotator as also envisaged herein will now be described with reference to FIGS. 1F and 1G. These Figures illustrate a particular embodiment of a rotator as envisaged herein.

FIG. 1F schematically illustrates a perspective view of a rotator 114. The rotator 114 is shaped like a hollow cylinder, which may be inserted and arranged e.g. on an inside of a housing of a device, and such that it either goes on an outside of a delivery member cover of the device, or on an inside of the delivery member cover of the device. The rotator 114 may for example include a groove (not shown) in its cylindrical wall which extends circumferentially, and which may interact with a protrusion of the housing of the device. Due to the alignment of such a groove, the interaction with the protrusion may allow the rotator 114 to rotate around its longitudinal axis L′, but not allow the rotator 114 to move along the longitudinal axis L′. In other embodiments, the protrusion may instead be part of the rotator 114 while the corresponding groove is part of the housing, still achieving the same effect.

The rotator 114 has a track 130 routed in its cylindrical wall, and the track 130 includes at least a first track edge 130a and a second track edge 130b. The first track edge 130a is tilted with an angle α with respect to a longitudinal axis L of the rotator, while the second track edge 130b is tilted with an angle β with respect to the longitudinal axis L. The track 130 is configured such that it can interact with a corresponding track follower of the device, when the track follower is received in the track 130 (i.e., when the track follower protrudes into the track 130). In particular, the angles α and β are such that the slopes of the track edges 130a and 130b are in opposite directions. Here, “opposite directions” should be understood as only meaning that the first angle α has an opposite sign to that of the second angle β, if both angles are measured the same way with respect to the longitudinal axis L′. Phrased differently, the angles should be such that independently on whether the first track edge 130a or the second track edge 130b is being pushed upon by the track follower, the rotation of the rotator 114 so caused is always in the same direction. In other words, the effect of this is that when the track follower engages (i.e. is pushed against) the first track edge 130a, the rotator 114 will rotate in a same direction as when the track follower instead engages (i.e. is pushed against) the second track edge 130b, even though the movement of the track follower while pushing on the first track edge 130a is in an opposite direction to that of the track follower while pushing on the second track edge 130b. This causes the rotator 114 to be movable between at least a first position (where the track follower can push against the first track edge 130a in one direction), a second position (where the track follower instead can push against the second track edge 130b if the movement of the track follower changes to an opposite direction), and also a third position wherein the track follower is not aligned with any one of the first track edge 130a and the second track edge 130b, such that pushing on the track follower does not bring the rotator 114 back to either its first position or its second position. This provides the same advantages as described earlier herein with reference to the devices 100 and 101 of FIGS. 1A-1E.

The rotator 114 also includes a first blocking member in form of an edge 150, where the edge 150 has a recess 152. This may interact (as previously described) with a corresponding second blocking member (i.e. a protrusion) of a release button of the device, thereby either blocking/limiting or allowing the release button from moving in a proximal direction of the device, depending on the current position of the rotator 114.

FIG. 1G schematically illustrates a perspective view of another embodiment of a rotator 114. The rotator 114 is the same as the rotator 114 described with reference to FIGURE IF, except that the first blocking member is now in form of a protrusion 140 which may interact with a corresponding second blocking member (an edge including a recess) to obtain the same limiting/allowing effect on the movement of the release button of the device. The blocking/limiting is then caused when an edge 141 of the protrusion 140 abuts the edge of the release button, and the allowing is caused when the protrusion 140 is aligned with the recess such that the edge 141 does not abut the edge of the release button when the release button is pushed towards the rotator 114.

In summary of FIGS. 1A-1G, it can be concluded that the various components of the devices 100 and 101 as envisaged herein are configured to interact such that the delivery member can be injected manually into the site of injection by pressing the delivery member cover against the injection site. The pressing of the delivery member cover against the injection site is also what eventually unlocks the release button such that it may be pushed to trigger the release of the medicament via the delivery member and into the injection site. More importantly, a device as envisaged herein can provide added safety functionality which, in particular situations, prevents the device from being triggered if the user e.g. only partly inserts the delivery member or e.g. forgets to push the release button before withdrawing the device from the injection site, and similar. The envisaged device(s) can also prevent a non-sterile delivery member from being accessible after such an event, by locking the delivery member cover and blocking it from being re-pushed in an attempt to restart the procedure of injection. Phrased differently, the user can be given a single opportunity to correctly perform the procedure of first inserting the delivery member by pushing the delivery member cover against the injection site, and then pushing the release button to trigger the delivery of the medicament. If the user fails performing such a procedure, further triggering of the device is prevented and an attempted reinsertion of the delivery member can also be blocked. This reduces any uncertainty that may arise due to such mishandling of the device, and also reduces the risks of exposing a non-sterile needle on which the user may be stinged.

Although not explicitly disclosed in any drawings herein, it is envisaged that the medicament delivery device may also include additional components, including e.g. a protective cap for the delivery members, an ampulla for storing the dose of medicament, one or more guiding rods and or other structures for the arrangement of the delivery member cover, the release button and the rotator as desired, a plunger rod and preloaded spring for, as part of the medicament delivery mechanism, force the medicament out of the ampulla/syringe when triggered, etc. For example, the medicament delivery mechanism may for example include a preloaded spring which, when released, forces a plunger rod to force the dose of medicament out through the delivery member and into the body of the user. The release of the preloaded spring may be caused in that a part of the release button, when the button is sufficiently pushed to cause such triggering, releases a holding mechanism for the spring, such that the spring may be free to exert a spring force on the plunger rod. For example, the spring may be held in a compressed/preloaded state by one or more gripping tabs. The release button may include a member which, when the release button is sufficiently pushed, interacts with the gripping tabs causing them to lose their grip of the spring. Other variants are of course also envisaged, as long as their overall functionality is the same as described herein.

Medicament delivery devices as envisaged herein may for example be autoinjectors containing insulin, allergy medicaments, epinephrine, migraine medicaments, atropine, or any other medicament/drug for which the use of a medicament delivery device as described herein is suitable. The device can e.g. be of a single-use type (i.e. disposable). Delivery members may include needles, parts of syringes, syrettes, or other suitable structures.

In the present disclosure, the term “longitudinal axis” of the device refers to an axis extending from a proximal end of the device to a distal end of the device, typically a central axis along the device in the direction of longest extension of the device. Likewise, “rotation”, “rotatably” an “rotational” refer to rotation relative to the longitudinal axis. The term “distal end” refers to the part/end of the device, or the parts/ends of the members thereof, which under use of the device is/are located furthest away from the dose delivery/injection site. Correspondingly, the term “proximal end” refers to the part/end of the device, or the parts/ends of the members thereof, which under use of the device is/are located closest to the dose delivery/injection site.

Although features and elements may be described above in particular combinations, each feature or element may be used alone without the other features and elements or in various combinations with or without other features and elements. Additionally, variations to the disclosed embodiments may be understood and effected by the skilled person in practicing the claimed invention, from a study of the drawings, the disclosure, and the appended claims.

In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

1-14. (canceled)

15. A medicament delivery device, comprising:
a release button;
a delivery member cover;
a rotator; and
a housing,
wherein the delivery member cover includes a track and the rotator includes a track follower, or vice versa, wherein the track follower protrudes into the track, and the track includes at least a first track edge and a second track edge tilted with a first angle and a second angle, respectively, with respect to a longitudinal axis of the device,
wherein the rotator includes a first blocking member and the release button includes a second blocking member,
wherein the delivery member cover is longitudinally moveable and rotationally fixed relative to the housing, the rotator is free to rotate relative to the housing, and the release button is longitudinally moveable and rotationally fixed relative to the housing,
wherein the rotator is configured to move from a first position to a second position to a third position, wherein the first position, second position and third position are all rotationally different from one another,
wherein in the first position of the rotator, the first track edge is aligned in a longitudinal direction with the track follower, and in the second position of the rotator, the second track edge is aligned in the longitudinal direction with the track follower,
wherein when the delivery member cover is moved in a distal direction relative to the rotator, the track follower engages the first track edge, thereby rotating the rotator from the first position to the second position in a first rotational direction, and wherein when the delivery member cover is subsequently moved back in a proximal direction relative to the rotator, the track follower engages the second track edge, thereby rotating the rotator from the second position to the third position in a second rotational direction,
wherein the first and second angles are such that the first and second rotational directions are the same,
wherein the first blocking member and the second blocking member are aligned in the first position of the rotator, thereby limiting movement of the release button in the proximal direction relative to the rotator,
wherein the first blocking member and the second blocking member are not aligned in the second position of the rotator, thereby allowing movement of the release button in the proximal direction relative to the rotator, and
wherein the first blocking member and the second blocking member are aligned in the third position of the rotator, thereby limiting movement of the release button in the proximal direction relative to the rotator.

16. The device according to claim 15, wherein in the first position of the rotator, the track follower is closer to a proximal end of the device than the first track edge, and wherein in the second position of the rotator, the track follower is closer to a distal end of the device than the second track edge.

17. The device according to claim 15, wherein, in the third position of the rotator, the delivery member cover is blocked from being moved in the distal direction relative to the rotator.

18. The device according to claim 17, wherein the delivery member cover includes at least one locking arm configured to, in the third position of the rotator, interact with a part of the device other than the delivery member cover to cause the blocking of the delivery member cover from being moved in the distal direction relative to the rotator.

19. The device according to claim 15, wherein in the third position of the rotator, neither the first track edge nor the second track edge is aligned in the longitudinal direction with the track follower.

20. The device according to claim 15, wherein the delivery member cover is spring loaded such that when the delivery member cover is moved in the distal direction relative to the rotator, potential spring energy is built up, and such that when the delivery member cover is subsequently moved back in the proximal direction relative to the rotator, this is at least partly driven by a release of said potential spring energy.

21. The device according to claim 15, wherein the first blocking member includes a protrusion and the second blocking member includes at least one edge including a recess, or vice versa, and wherein:
in the first and third positions of the rotator, the protrusion rotationally aligns with the at least one edge but not with the recess, and
in the second position of the rotator, the protrusion rotationally aligns with the recess.

22. The device according to claim 21, wherein the at least one edge is at a same position relative to the longitudinal axis on both sides of the recess.

23. The device according to claim 15, further including means for either directly or indirectly holding a delivery member in form of a needle for the delivery of the medicament, and configured such that the delivery member can be manually inserted into a body by a user of the device moving the delivery member cover in the distal direction relative to the rotator by pressing the delivery member cover against an injection site on the body.

24. A rotator for a medicament delivery device including a release button, a delivery member cover and a housing, wherein the rotator has a longitudinal axis and includes a track configured to receive a track follower of the delivery member cover, and at least a first track edge and a second track edge tilted with a first angle and a second angle, respectively, with respect to the longitudinal axis,
wherein, when the rotator is arranged in the device to freely rotate relative to the housing and with the track follower received in the track, the rotator is configured to move from a first position to a second position to a third position, wherein the first position, second position and third position are all rotationally different from one another,
wherein in the first position of the rotator, the first track edge is aligned in the longitudinal direction with the track follower, and in the second position of the rotator, the second track edge is aligned in the longitudinal direction with the track follower,
wherein when the delivery member cover is moved in a distal direction relative to the rotator, the track follower engages the first track edge, thereby rotating the rotator from the first position to the second position in a first rotational direction, and wherein when the delivery member cover is subsequently moved back in a proximal direction relative to the rotator, the track follower engages the second track edge, thereby rotating the rotator from the second position to the third position in a second rotational direction, and
wherein the first and second angles are such that the first and second rotational directions are the same.

25. The rotator according to claim 24, including a first blocking member configured to interact with a second blocking member of the release button to either limit or not limit movement of the release button in the proximal direction relative to the rotator depending on the position of the rotator.

26. The rotator according to claim 25, wherein the first blocking member includes a protrusion configured to interact with a corresponding at least one edge including a recess of the second blocking member of the release button.

27. The rotator according to claim 25, wherein the first blocking member includes at least one edge including a recess configured to interact with a corresponding protrusion of the second blocking member of the release button.

28. The rotator according to claim 27, wherein the at least one edge is at a same position relative to the longitudinal axis of the rotator on both sides of the recess.
